(19) United States
(12) Patent Application Publication
(10) Pub. No.: US 2020/0276341 A1
(43) Pub. Date: Sep. 3, 2020

(54) Heat sterilizer

(71) Applicant: Henny Jakobsgaard, Hellerup (DK)

(72) Inventor: Lars Schøning, Hellerup (DK)

(21) Appl. No.: 16/646,203

(22) PCT Filed: Sep. 11, 2018
(86) PCT No.: PCT/DK2018/000095

§ 371 Date: Mar. 11, 2020

(30) Foreign Application Priority Data
Sep. 13, 2017 (DK) PA 2017 70690

(51) Int. Cl. A61L 2/00; A61B 50/33; A61L 2/26

(52) U.S. Cl. CPC A61L 2/0023; A61B 2050/005; A61L 2/26; A61B 50/33

(57) ABSTRACT

By designing a heat sterilizer in the form of an oven having heating elements and a cassette of metal containing the objects to be sterilized placed in the cassette, and where the inside of same is provided with a coating with a high emissivity, the sterilization may take place both at field hospitals and clinics and at stationary hospitals since the equipment is light and moreover only has a modest power consumption.

### PRIOR ART

The invention relates to a heat sterilizer comprising an oven wherein a cassette can be positioned with a view to heating, which cassette may hold the instruments, materials to be sterilized.

Sterilization of particularly surgical instruments usually takes place in an autoclave wherein the sterilization is done by means of a steam pressure of up to 3 atm. This may impart a maximum temperature of up to 140′C to the instruments.

This autoclave equipment has a substantial weight and therefore it is normally only used in the form of fixedly installed autoclaves. It is a further drawback that not all supplies such as bandages etc. can stand this type of heating without being damaged or will subsequently have to be dried.

Moreover, sterilization by means of lamps emitting ultra-violet rays are known but in such cases the sterilization is confined to the directly irradiated surfaces.

Finally, sterilization by means of ozone is known which, however, is a toxic gas and accordingly requires special safety measures.

### OBJECT OF THE INVENTION

It is the object of the invention to remedy these deficiencies and drawbacks and at the same time to provide a light and so mobile sterilization equipment.

This is achieved according to the invention by means of a cassette which can be positioned in an oven space, said cassette comprising a tray with a lid made of metal and provided with a coating on the inside, said coating comprising materials with a high emissivity.

In this way, it is possible to sterilize for example surgical equipment and appurtenant utilities solely by means of dry heat and as the emission through the walls of the cassette is very high, it will be possible to reach temperatures up to as high as 249° C.

This will reduce the heating time just as it will be possible to manufacture ovens and cassettes in the nature of small mobile units making the equipment suitable for field applications.

By using electrical heating elements as a heat source as mentioned in claim 2, the provision of a high-power compact oven having an insignificant heat loss by the heat transmission is ensured.

By using electrical heating elements in the form of either resistance and/or induction heating elements as mentioned in claim 3, it is possible to select an economical and practical heating of the cassette.

By being able to displace the heating elements as mentioned in claim 4, partly for tight abutment on the surface of the cassette and partly away from same, the cassette may easily be inserted in and be removed from the oven after sterilization.

By using either a stationary power supply or a mobile accumulator-based power source as mentioned in claim 5, mobile application of the sterilizer is ensured.

Finally, as mentioned in claim 6, it is advantageous to use the sterilizer in big as well as in small surgical units.

### DRAWING

An example of a preferred embodiment will be further described in the following with reference to the drawing wherein

FIG. 1 is a section through a cassette and its contents,

FIG. 2 is an enlarged sectional view of the wall/lid with coatings, and

FIG. 3 is a sectional view of an oven with an inserted cassette.

### DESCRIPTION OF AN EXAMPLE OF AN EMBODIMENT

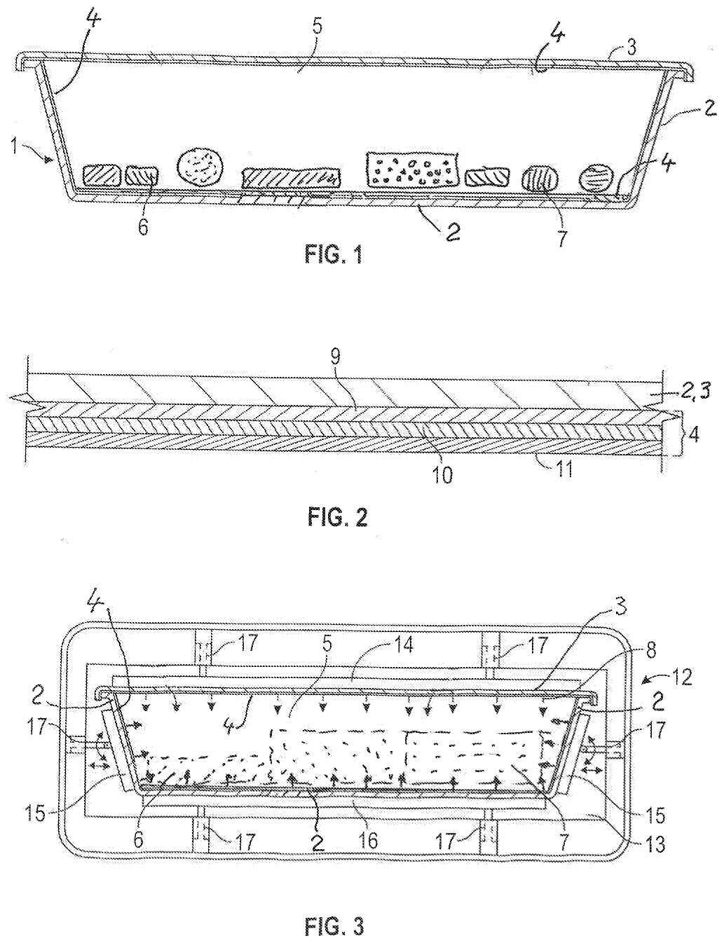

FIG. 1 shows an example of an embodiment of a cassette 1 comprising a tray 2 having a lid 3. These parts are all made of metal such as stainless steel, aluminium or other metals and alloys.

The instruments and other objects 6, 7 may be placed in this cassette with a view to subsequent sterilization in an oven.

As shown in FIG. 2, which is an enlarged sectional view of the basic material 2, 3, the same is provided with a coating 4 in the form of a laminate.

This coating consists of at least two different layered materials 9 and 10. The materials may comprise pulverized, micronized substances having high emissivity and/or high refractory indexes.

Examples of such suitable substances are Ga with an index of about 4.0, GaP of about 3.0, SiC of about 2.5, TiO₂ of about 2.4 and GaN with an index of about 2.3. Morever, Carbon Black and Vantablack may be mentioned as examples of materials with a high emissivity.

These materials are applied in layers for forming a laminate 4 in that on the basic material 2, 3 there is applied a first layer 9 for example by printing, rolling or the like of a pulverized material dissolved in a binding agent where after a further layer 10 may be applied.

These two layers preferably have different refractions for forming an efficient internal reflection by the refraction of the heat waves between the layers.

Furthermore, a third layer 11 may be applied which may consist of e.g. ZnS or TiO₂, the white colour thereof providing an attractive appearance.

Heating of cassette 1 with its contents 6, 7 takes place in an oven 12 with an oven chamber 13 as shown in FIG. 3.

FIG. 3 shows a sectional view of an oven 12 comprising an oven chamber 13. In this chamber 13 there are in this case arranged heating elements 14, 15, 16 which may abut on the surface of the cassette with its contents 6 and 7.

A heating element 16 is arranged below, four heating members 15 along the side surfaces of the cassette and an upper heating element 14 may abut on the lid 3.

As indicated by arrows 8, the heat stress will be equally directed towards the contents 6, 7 in the cassette.

To facilitate the insertion as well as the removal of the tray, some of or all heating elements are displaceable towards and away from the cassette by means of actuators 17 which may also be pivotable as indicated by arrows.

Either resistance or induction heating elements may be used as a power source and same may be both a fixed installation with normal line voltage or a low-voltage power source such as an accumulator-based power source may be used.

The result is a mobile, i.e. movable, sterilization unit which can be used e.g. in connection with field hospitals or clinics.

Since the oven may be manufactured to have various sizes, it will be possible to use various sizes of cassettes providing for manufacturing of relevant cassette sizes and ovens according to requirements.

The surface coating 4 is non-toxic and will in no way emit harmful gases so that the sterilization will comply with all requirements on sterilization of surgical equipment and the like.

1. A heat sterilizer comprising an oven wherein a cassette can be positioned with a view to heating, which cassette may hold the instruments, materials to be sterilized, characterized in that the cassette (1) comprises a tray (2) having a lid (3) made of metal and provided with a coating (4) on the inside, said coating comprising materials with a high emissivity.

2. The heat sterilizer according to claim 1, characterized in that the oven (12) is provided with electrically heated contact elements (14, 15, 16) which elements may abut on the tray (2) and lid (3) of the cassette (1).

3. The heat sterilizer according to claim 2, characterized in that the contact elements (14, 15, 16) comprise resistance heating elements and/or induction heating elements.

4. The heat sterilizer according to claim 2, characterized in that the contact elements (14, 15) are displaceable and/or turnable relative to the tray (2) and lid (3) of the cassette (1) with a view to easy insertion and removal of the cassette (1) following the heat treatment.

5. The heat sterilizer according to claim 2, characterized in that the power source of the elements (14, 15, 16) may be either stationary or mobile power supply.

6. Use of the heat sterilizer mentioned in claim 1 for use in hospitals and clinics both stationary and mobile such as field and emergency clinics.

7. The heat sterilizer according to claim 3, characterized in that the contact elements (14, 15) are displaceable and/or turnable relative to the tray (2) and lid (3) of the cassette (1) with a view to easy insertion and removal of the cassette (1) following the heat treatment.

8. The heat sterilizer according to claim 3, characterized in that the power source of the elements (14, 15, 16) may be either stationary or mobile power supply.

9. The heat sterilizer according to claim 4, characterized in that the power source of the elements (14, 15, 16) may be either stationary or mobile power supply.
